(19) United States
(12) Patent Application Publication
(10) Pub. No.: US 2003/0181986 A1
(43) Pub. Date: Sep. 25, 2003

(54) Surgical element

(76) Inventor: Jurgen Buchholz, Heidelberg (DE)

Correspondence Address: BRUCE LONDA, NORRIS, MCLAUGHLIN & MARCUS, P.A., 220 EAST 42ND STREET, 30TH FLOOR, NEW YORK, NY, 10017, US

(21) Appl. No.: 10/343,298

(22) PCT Filed: Jul. 23, 2001
(86) PCT No.: PCT/EP01/08481

(30) Foreign Application Priority Data
Jul. 29, 2000 (DE) 10037345.3

(51) Int. Cl. A61F 2/32

(52) U.S. Cl. 623/22.12

(57) ABSTRACT

Surgical application, holding, fixing and/or fastening element with a shaft section that is elongated in at least certain sections, for example hip endoprosthesis (10) with a shaft (12) to be secured in a medullary channel of the femur of a patient, pins or bone screws for pinning fractures and/or fixing the position of bone fragments after bone surgery. The elongated shaft section includes a through bore (20) extending essentially in the longitudinal direction of the shaft section (12) and being open-ended on at least one end for introducing a flowable mass that hardens or sets after introduction, and at least one through opening (22) that connects the through bore with the outside of the shaft section.

An elongated sleeve-like or tubular distribution element (24) which at least partially contacts the wall of the through bore (20) and which is moveable and/or rotatable in the through bore, is arranged in the through bore (20). The distribution element (24) is provided with at least one through opening (26) which can be aligned or moved out of alignment by moving and/or rotating the distribution element (24) relative to a respective one of the through openings (22) in the shaft section (12).

The invention relates to a surgical application, holding, fixing and/or fastening element with a shaft section that is elongated in at least certain sections, for example a hip endoprosthesis with a shaft to be secured in a medullary channel of the femur of a patient, pins or bone screws for pinning fractures and/or fixing the position of bone fragments after bone surgery etc., wherein the elongated shaft section includes a through bore extending essentially in the longitudinal direction of the shaft section and being open-ended on at least one end for introducing a flowable mass that hardens or sets after introduction, and at least one through opening that connects the through bore with the outside of the shaft section.

Surgical instruments of this type with shaft sections that are secured either in naturally occurring bone channels, such as the medullary channel of the femur or in bores produced by an operator with surgical tools, and that are used to fix either prosthesis parts stable enough to later carry a load or to fix bone segments after bone fractures with a predetermined mutual alignment, are successfully used particularly in orthopedic surgery on a large scale. However, the application of such surgical aids is problematic in situations where the bone segment which is to receive the shaft section has a reduced load-carrying capacity. In particular, in older patients afflicted with osteoporosis, but also in younger patients with comminuted fractures, the application of pins—for example for treating fractures of the femural neck or for fixing the shaft of a hip joint prosthesis in the medullary channel—can cause problems. In particular in osteoporosis patients, the load-carrying capacity of the bone, particularly in cancellous bone regions, can be reduced to a point where a bone screw cannot be secured with sufficient load-carrying capacity in the bone. In these situations, the affected limbs have to be immobilized so as to keep loads off the attachment location, until new bone tissue has formed to provide the required stability. Disadvantageously, a joint immobilized in this matter may become stiff, so that this joint may no longer be fully or even partially functional after removal of the dressing, although the bone fracture has healed. This applies also to comminuted fractures, even in younger patients, where it is important to prevent soft scar tissue from growing in the gaps and cavities between the bone fragments after the fracture has been set by introducing a medullary pin and the bone fragments have been aligned. It is important that after the fracture is set, edema accumulating in the cavities or gaps is displaced and the cavities or gaps are filled with a material that prevents the growth of soft tissue. The material introduced in the cavities and/or gaps can be bone cement which is malleable at first before hardening or setting. More recently, materials have advantageously been used that can break down in the body after hardening or setting, i.e., that are resorbed by the body over time. These materials have a composition which allows the resorption to take place over a time that corresponds at least approximately to the time needed for newly formed bone cells to grow through the aforedescribed cavities. In particular, plaster of Paris which is paste-like and flowable when prepared with water in a sterile environment can be used under certain processing conditions, where it can be introduced directly at the defected locations using needles or syringes without opening the surrounding soft tissue area. The plaster can harden and fill the defected locations and fix the position of bone fragments. The region of a comminuted fracture is thereby stabilized in addition to the actual metallic medullary pin or a bone screw, with the existing cavities being filled in through introduction of the hardening plaster material, which displaces the edema and prevents the growth of soft tissue in the cavities. The plaster is then relatively quickly resorbed by the body and replaced by newly formed bone tissue. This stabilizes the region of the fracture very quickly which shortens significantly the time required for an additional external plaster cast or—as for joint prostheses—for an immobilization of the joint region. The risk that the joints become stiff is also significantly reduced, since the joint can be moved again after a relatively short time after surgery. The same applies for securing bone screws in damaged cancellous bone regions. In this case, these regions should be filled with the hardening or setting resorbable mass immediately before, during or immediately after the bone screws are screwed in, so that a load-carrying bone region, in which the thread of the bone screw can hold, is formed after the mass has hardened. The hardened material is then again broken down in the body by resorption and, like in the ideal case, replaced by the same quantity of load-carrying bone tissue.

Advantageously, as contemplated above, paste-like masses, i.e., masses which can be processed in a flowable state and which set or harden and are resorbed in the body, can also be used to affix prosthesis shafts in the medullary channel, instead of conventional bone cement which is not resorbed, but merely fills the gap between the shaft of the prosthesis and the wall of the medullary space.

A solution of the aforedescribed problem assumes that during the surgery when a prosthesis is inserted or a fracture is set, the setting resorbable material can be introduced in the anchoring region of necessary prosthesis shafts, medullary space pins or bone screws under sterile conditions in the stabilizing region, without having to surgically open the patient's soft tissue surrounding the fracture location.

It is therefore an object of the invention to form surgical elements used for stabilizing fractures and/or fixing prostheses in such a way that the resorbable setting material can be introduced—with the same surgical aids that are used for attachment—directly under pressure into the region of the bone that is to be reinforced, regardless if the surgical aid remains in the bone only temporarily or permanently, or if the surgical aid is introduced into the region to be reinforced only during the introduction process of the setting material and later removed.

The object is solved by the invention with surgical elements of the aforedescribed type, in that there is arranged in the through bore an elongated sleeve-like or tubular distribution element which at least partially contacts the wall of the through bore and which is moveable and/or rotatable in the through bore, wherein the distribution element is provided with at least one through opening which can be aligned or moved out of alignment by moving and/or rotating the distribution element with respect to a corresponding one of the through openings in the shaft section. With this arrangement, the setting or hardening mass that is prepared from the components in a mixing and application device, can be introduced under pressure into the interior space of the distribution element and thereafter expelled into the adjacent bone region through one or several through openings in the distribution element and a through opening in the shaft section that is aligned with these through openings. By rotating or displacing the sleeve element, the through openings of the distribution element and the shaft section can be aligned for passage of the still paste-like mass or can be offset with respect to each other, so that the subsequently hardening mass can be directly applied to the desired bone regions.

In an advantageous embodiment of the invention, the sleeve-like or tubular distribution element is guided out of the open end of the through bore and openly terminates at this end, so that the above-mentioned mixing and application devices can be easily connected to the distribution element.

Depending on the specific application, the sleeve-like or tubular distribution element arranged in the through bore can be closed or open on the end opposite the open end of the through bore.

The open end or the two open ends of the sleeve-like or tubular distribution element is/are each provided with an adapter for connection to an application device which supplies a flowable mass under pressure and/or to a vacuum source. If a distribution element that is open on both sides is used, then the adapter provided on the outer end can be connected to the application device supplying the flowable mass under pressure, and the opposing inner adapter can be connected via suitable connecting lines or channels to the vacuum source. Blood and edema residing in the application area can be suctioned off with the vacuum source before the flowable mass is introduced.

If a distribution element that is open only on one side is used, then the distribution element can be subdivided into two separate channels, wherein each of the two channels disposed at the open end of the distribution element can include an adapter for connection to, on one hand, an application device supplying a flowable mass and, on the other hand, a vacuum source.

Advantageously, markings can be provided in the region of the open mouth of the through bore of the surgical element and in the associated end region of the sleeve-like or tubular distribution element for indicating the relative displacement and/or rotation of the distribution element in the through bore. These markings can be used for adjusting the alignment and/or offset of the through openings in the distribution element and the shaft section, whereby a specific region can be selected where the setting mass to be applied exits the shaft section.

If the shaft section has several through openings that are mutual offset in the longitudinal direction of shaft, then the elongated sleeve-like or tubular distribution element can have a plurality of through openings, with the number and the relative position of these through openings corresponding to the number and the relative position of the through openings in the shaft. The through openings in the shaft section and in the distribution element can hence be simultaneously aligned or misaligned, so with the surgical element constructed in this manner, the simultaneously introduced flowable mass can be distributed over the length of the shaft section.

Alternatively, the distribution element can have a plurality of through openings, with the number of the through openings in the distribution element corresponding to the number of through openings in the shaft section, whereby the relative position of the through openings in the distribution element deviates from the relative position of the through openings in the shaft section in such a way, that only one of the through openings or only a portion of the through openings in the distribution element are aligned with the through bores in the shaft section. The through bores in the shaft section and the distribution element that are not aligned with each other can be aligned, if necessary, by rotating or displacing the distribution element in the through bore of the shaft section. However, the previously aligned through bores are then moved out of alignment. This allows sequential introduction of a hardening or setting mass in different bone regions.

In an advantageous embodiment of the invention, the distribution element is formed as a thin-wall metal tube at least in the region intended to be mounted in the through bore in the shaft section. Alternatively, the region located in the through bore can also be formed as an elastically bendable tube which due to its elastic deformability can adapt to through bores that are not exactly straight.

Such an elastic tube can preferably be made of plastic which can be reinforced with a fiber or fabric insert.

The invention will be described in detail hereinafter with reference to an embodiment illustrated in the appended drawing, which shows in:

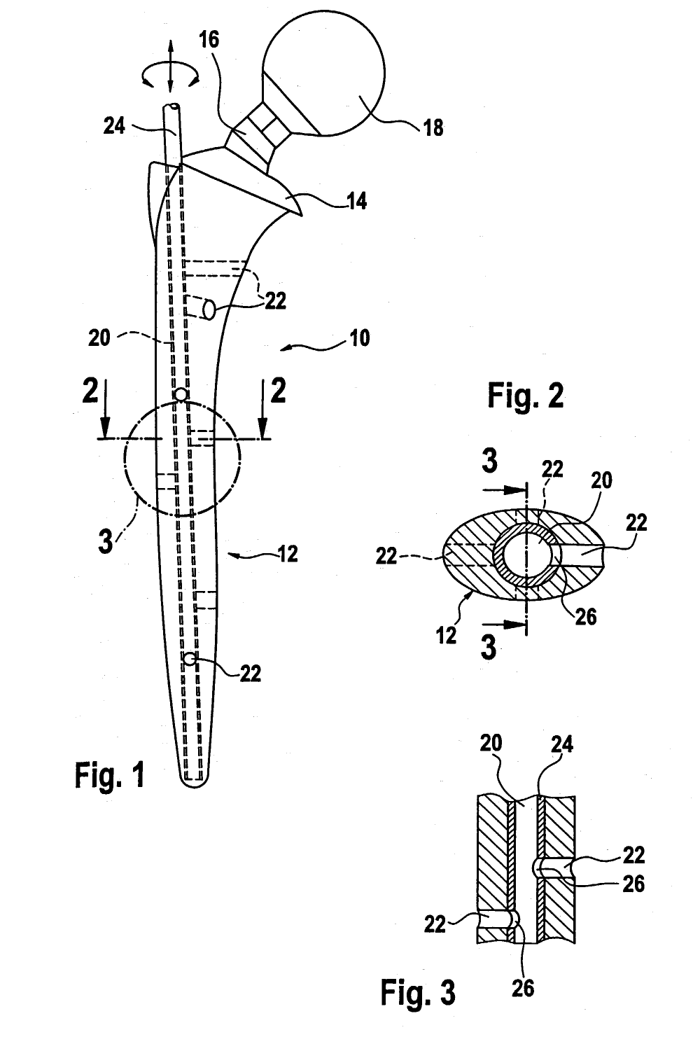

FIG. 1 a side view of a hip joint endoprosthesis formed according to the invention;

FIG. 2 an enlarged cross-sectional view through the shaft of the prosthesis taken along the line indicated in FIG. 1 by the arrows 2-2; and

FIG. 3 a cross-sectional view through the section of the shaft of the endoprosthesis located inside the region 3 surrounded by the dash-dotted line of FIG. 1 and cut in a cutting plane indicated in FIG. 2 by the arrows 3-3.

The embodiment according to the invention illustrated in the drawing and indicated in its entirety with the reference numeral 10, is a hip joint endoprosthesis with an elongated shaft 12 formed as a cement shaft typical for such prostheses, which can be inserted conventionally into the medullary channel of a femur that has been surgically prepared in a proximal end region and secured with bone cement. Conventionally, after application of a medullary plug at a suitable depth and before insertion of the shaft 12 into the corresponding widened medullary channel, a specified quantity of bone cement is filled in, whereafter the shaft of the endoprosthesis is inserted to the predetermined position, i.e., until the radially enlarged collar 14 contacts the contact surface prepared at the end of the femur. After the bone cement has set, the shaft 12 and hence also the spherical ball 18 of the prosthesis affixed to the shaft are firmly anchored in the femur.

However, in practice, with this kind of attachment of shaft 12 in the medullary channel, air bubbles may still remain trapped in the bone cement which weaken the joint between the shaft and the femur. A through bore 20 therefore extends over the entire length of the shaft 12 of the endoprosthesis according to the invention, with through openings 22, which are offset in the longitudinal direction and are oriented in different directions, extending from the through bore 20 to the outside of the shaft 12. An elongated hollow distribution element 24 which is tubular at least in the region located in the through bore 20, is inserted in the through bore 20, with the fit between the outside of the distribution element 24 and the wall of the through bore 20 being selected so that the distribution element can be rotated and/or displaced in the through bore. The distribution element 24 also includes through openings 26 which—depending on the relative rotation or longitudinal positioning of the shaft 12 in the through bore 20—can be either aligned with corresponding through openings 22 of the shaft 12 or offset from the through openings 22. It is apparent that a flowable mass that later sets or hardens, e.g., bone cement, can be pressed under pressure by using a suitable (conventional) application device through the end of the distribution element 24, which is guided out of the upper end of the through bore 20, into the distribution element and then into the medullary channel through the aligned through openings 26 and 22. The introduction of the mass can be controlled by suitably aligning or misaligning of the through openings 26, 22 with respect to each other, so that the bone cement can initially exit from the tip of the shaft and subsequently fill the cavities between the outer wall of the shaft and the medullary channel by ascending towards the open end. Any air remaining in the cavities is displaced towards the open end of the femur. The consecutive through openings 22, 26 in the longitudinal direction of the shaft can be aligned by controlling the rotation position and/or by a longitudinal adjustment of the distribution element 24 in the through bore 20, so that the bone cement introduced under pressure completely fills the space between the shaft and the medullary channel of the femur, without air inclusions, as soon as the bone cement filling has reached the respective bore. A secure and load-bearing seating of the prosthesis in the medullary channel is guaranteed after hardening of the bone cement. The remaining hardened bone cement provides an additional formfitting attachment of the shaft in the medullary channel through a material connection of the outside of the shaft with the femur. The distribution element 24 can remain in the through bore 20, with the upper end section of the distribution element projecting from the shaft being cut off after introduction of the bone cement. Alternatively, the distribution element 24 can also pulled out of the through bore 20 after introduction of the bone cement, but before the bone cement has completely hardened.

The relative position of the through openings 22 and 26 in the shaft 12 and the distribution element 24, respectively, can be monitored by applying markings in form of a ruler on the outside of the distribution element, which can then be aligned with a marking applied on the outside of the upper shaft end, thereby indicating the relative alignment of the through openings 26 and 22.

The main function described above in conjunction with the aforedescribed exemplary embodiment of a hip joint endoprosthesis 10 can also be extended to other surgical elements, such as pins, bone screws and the like. Here, too, a through bore with branching through openings is provided in the shaft portion of the corresponding element to be secured in the bone, wherein an associated sleeve-like or tubular distribution element with through openings and an adjustable position is inserted in the through bore. By relative rotation or longitudinal displacement of the distribution element in the shaft portion, the through openings in the shaft portion and the distribution element can be selectively aligned or misaligned. When such pins or bone screws are used for treating bone fractures in patients with osteoporosis or comminuted fractures, a flowable material which hardens after introduction can be applied instead of bone cement, wherein the flowable material is slowly resorbed or broken down in the body concurrent with the growth of bone cells in the cavities produced by resorption.

Needles can also be designed for introducing resorbable materials that harden or set. The hardening or setting material can be introduced preventively and before surgical procedures through the needles into bone regions that are prone to fracture, thereby producing a more solid seat with improved load-bearing capacity for conventional pins or screws that are subsequently inserted for treating the fractures. In situations where blood, edema and the like accumulate in the bone regions to be stabilized by introduction of flowable, hardening or setting materials, the needle can also be configured so that blood and/or edema are suctioned off by connecting the inside of the needle to a vacuum source, before the flowable, settable or hardening material is introduced through the outer open end.

1. Surgical application, holding, fixing and/or fastening element with a shaft section that is elongated in at least certain sections, for example a hip endoprosthesis (10) with a shaft (12) to be secured in a medullary channel of the femur of a patient, pins or bone screws for pinning fractures and/or fixing the position of bone fragments after bone surgery etc., wherein the elongated shaft section (12) includes a through bore (20) extending essentially in the longitudinal direction of the shaft section and being open-ended on at least one end for introducing a flowable mass that hardens or sets after introduction, and at least one through opening (22) that connects the through bore (20) with the outside of the shaft section (12),
characterized in
that there is arranged in the through bore (20) an elongated sleeve-like or tubular distribution element (24) which at least partially contacts the wall of the through bore (20) and which is moveable and/or rotatable in the through bore, which distribution element (24) is provided with at least one through opening (26) which can be aligned or moved out of alignment by moving and/or rotating the distribution element (24) with respect to one of the through openings (22) in the shaft section (12).

2. The surgical element of claim 1, characterized in that the sleeve-like or tubular distribution element (24) is guided out of the open end of the through bore (20) and openly terminates at this end.

3. The surgical element of claim 1 or 2, characterized in that the sleeve-like or tubular distribution element (24) arranged in the through bore is closed on the end opposite the open end of the through bore (20).

4. The surgical element of claim 1 or 2, characterized in that the sleeve-like or tubular distribution element (24) arranged in the through bore is open on the end opposite the open end of the through bore (20).

5. The surgical element of claim 3 or 4, characterized in that the open end or the two open ends of the sleeve-like or tubular distribution element (24) is/are each provided with an adapter for connection to an application device which supplies a flowable mass under pressure and/or to a vacuum source.

6. The surgical element of claim 3, characterized in that the elongated sleeve-like or tubular distribution element (24) is subdivided into two mutually separated channels, and that on each of the two channels at the open end of the distribution element there is provided an adapter for connection to, on one hand, an application device supplying a flowable mass and, on the other hand, a vacuum source.

7. The surgical element according to one of the claims 1 to 6, characterized in that, on one hand, in the region of the open mouth of the through bore (20) of the surgical element (10) and, on the other hand, in the associated end region of the sleeve-like or tubular distribution element (24) there are provided markings which indicate the relative displacement and/or rotation position of the distribution element in the through bore.

8. The surgical element according to one of the claims 1 to 7, wherein the shaft segment (12) includes a plurality of through openings (22) which are mutually offset in the longitudinal direction of the shaft, characterized in that in the elongated sleeve-like or tubular distribution element (24) there are provided a plurality of through openings (26), with the number and the relative position of the through openings (26) corresponding to the number and the relative position of the through openings (22) in the shaft (12).

9. The surgical element according to one of the claims 1 to 7, wherein the shaft segment includes a plurality of through openings which are mutually offset in the longitudinal direction of the shaft, characterized in that the distribution element (24) includes a plurality of through openings (26), which corresponds in number to the number of through openings (22) in the shaft section (12), which however deviate in their relative mutual position from the relative position of the through openings (22) in the shaft section (12) in such a way that an alignment of the through bores (26; 22) in the distribution element (24) and in the shaft section (12) can be adjusted only with respect to one of the through openings (26; 22) or a portion of the through openings (26; 22).

10. The surgical element according to one of the claims 1 to 9, characterized in that the distribution element (24) is formed as a thin-wall metal tube at least in the region intended to be mounted in the through bore (20) in the shaft section (12).

11. The surgical element according to one of the claims 1 to 9, characterized in that the distribution element (24) is formed as a thin-wall elastically bendable tube at least in the region intended to be mounted in the through bore (20) in the shaft section (12).

12. The surgical element of claim 11, characterized in that the section of the distribution element (24) formed as a thin-wall elastically bendable tube is made of plastic.

13. The surgical element of claim 12, characterized in that the material of the section of the distribution element (24) formed as a plastic tube is reinforced with fiber or fabric inserts.
